(19) United States
(12) Patent Application Publication
(10) Pub. No.: US 2025/0163097 A1
(43) Pub. Date: May 22, 2025

(54) PROCESS FOR THE BACKBONE DEPROTECTION OF OLIGONUCLEOTIDES CONTAINING A TERMINAL ALKYL PHOSPHONATE GROUP

(71) Applicant: Dicerna Pharmaceuticals, Inc., Lexington, MA (US)

(72) Inventors: Marek Stanislaw KOMISARSKI, Basel (CH); Pascal MUELLER, Basel (CH); Martin OLBRICH, Basel (CH); Johanna ROMANO, Basel (CH); Andreas SCHUSTER, Basel (CH)

(21) Appl. No.: 18/723,131

(22) PCT Filed: Dec. 22, 2022
(86) PCT No.: PCT/US2022/082239

§ 371 Date: Jun. 21, 2024

(30) Foreign Application Priority Data
Dec. 22, 2021 (EP) 21216879.3

(51) Int. Cl. C07H 21/00; C07H 1/00

(52) U.S. Cl. CPC C07H 21/00; C07H 1/00

(57) ABSTRACT

The invention comprises a process for the production of a linear P-linked oligonucleotide bearing an alkyl phosphonate group at 5′-terminus of the formula I

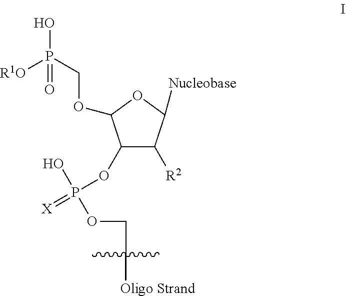

wherein R¹ is a C₁₋₄-alkyl group, R² is fluoro, hydroxyl or C₁₋₄-alkoxy, X is sulfur or oxygen, the term Nucleobase stands for an optionally modified adenine, cytosine, guanine or uracil and the term oligo strand stands for the remaining P-linked oligo nucleotide strand, and wherein the oligo strand comprises at least one uracil nucleobase.

The process is performed under conditions that the level of alkyl transfer impurities and the level of cyanoethyl (CNET) impurities in the linear P-linked oligonucleotide of formula I, is substantially reduced.

### CROSS-REFERENCE TO RELATED APPLICATIONS

This application claims the benefit of EP Patent Application No. 21216879.3, filed on Dec. 22, 2021, the entirety of which is incorporated herein by reference thereto.

### SEQUENCE LISTING

The instant application contains a Sequence Listing which has been submitted electronically in XML format and is hereby incorporated by reference in its entirety. Said XML copy, created on Jan. 10, 2025, is named 0243_0039-PCT-US_SL.xml and is 28,778 bytes in size.

The invention relates to a novel process for the production of a linear P-linked oligonucleotide bearing an alkyl phosphonate group at the 5′-terminus of the formula I

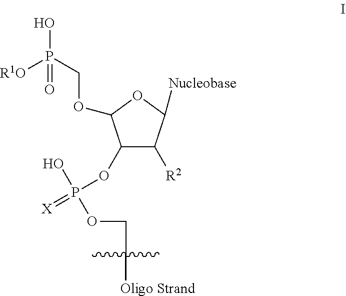

wherein R1 is a C1-4-alkyl group, R2 is fluoro, hydroxyl or C1-4-alkoxy, X is sulfur or oxygen, the term Nucleobase stands for an optionally modified adenine, cytosine, guanine or uracil and the term oligo strand stands for the remaining P-linked oligonucleotide strand and wherein the oligo strand comprises at least one uracil nucleobase.

P-linked oligonucleotides bearing an alkyl phosphonate group at the 5′-terminus of the formula I are therapeutically valuable compounds, for example, as antisense strands, can target various mRNAs and thereby block the expression of the corresponding genes. As an example P-linked oligonucleotides bearing an alkyl phosphonate group at the 5′-terminus of the formula I can be antisense strands targeting the HBsAg mRNA that translate HBV surface antigens (HBsAg), effecting knock down of the HBsAg gene expression and accordingly may be useful to treat HBV infections (International Publication WO 2019/079781).

The process comprises the removal of the cyanoethyl group and one C1-4 alkyl group from the oligonucleotide compound of formula II

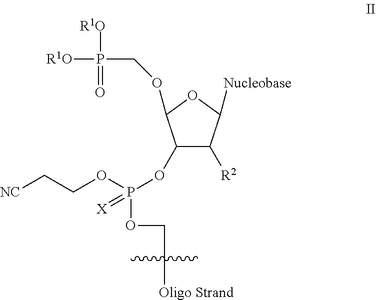

wherein R1, R2, X, the term Nucleobase and the term oligo strand is as above, with a nucleophilic organic base in the presence of an organic solvent.

The oligonucleotide synthesis in principle is a stepwise addition of nucleoside residues to the 5′-terminus of the growing chain until the desired sequence is assembled.

As a rule, each addition is referred to as a synthetic cycle and in principle consists of the chemical reactions
a₁) de-blocking the 5′protected hydroxyl group on the solid support,
a₂) coupling the first nucleoside as an activated phosphoramidite with the free hydroxyl group on the solid support,
a₃) oxidizing or sulfurizing the respective P-linked nucleoside to form the respective phosphodiester (P═O) or the respective phosphorothioate (P═S);
a₄) optionally, capping any unreacted hydroxyl groups on the solid support,
a₅) de-blocking the 5′ hydroxyl group of the first nucleoside attached to the solid support;
a₆) coupling the second nucleoside as activated phosphoramidite to form the respective P—O linked dimer;
a₇) oxidizing or sulfurizing the respective P—O linked dinucleoside to form the respective phosphodiester (P═O) or the respective phosphorothioate (P═S);
a₈) optionally, capping any unreacted 5′ hydroxyl groups;
a₉) repeating the previous steps as to as until the desired sequence is assembled.

The reaction sequence may alternatively start with de-blocking of the 5′ protected hydroxyl group of the nucleoside which is preloaded on the solid support. The subsequent steps follow the sequence as outlined above.

Finally, the assembled oligonucleotide is treated with an organic soluble base to remove the cyanoethyl protecting group, treated with aqueous base to effect a global base deprotection and cleavage from the solid support (commonly referred to as cleavage and deprotection), and finally subjected to subsequent downstream processing and purification methods to provide the desired pure oligonucleotide.

The backbone deprotection i.e. the removal of the cyanoethyl group from the phosphodiester or phosphorothioate linkage is standard in principle and is well known in the art.

The U.S. Pat. No. 6,887,990 illustrates the standard procedure and discloses the removal of cyanoethyl groups with an amine such as diethylamine in an acetonitrile solution.

The removal of one alkyl group from an oligonucleotide compound of formula II has been described with concentrated ammonia at 55° C. in the PCT International Publication WO 2018/045317.

It has been observed that in the course of the process the alkyl group has a tendency to alkylate uracil nucleobases in proximity to the alkyl phosphonate nucleotide, which results in so called alkyl transfer impurities which cannot be significantly depleted in the subsequent downstream processing steps. An oligo structure with a methylated uracil nucleobase is shown in formula III. R2 has the meaning as outlined before.

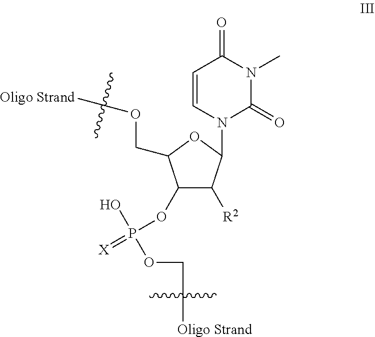

Object of the present invention, therefore, was to further improve the process for the backbone deprotection, i.e. for the removal of the cyanoethyl group and one C1-4 alkyl group from the oligonucleotide compound of formula II. Particularly the task was to find reaction conditions which suppress or at least reduce unwanted side reactions, such as the formation of alkyl transfer impurities and of cyanoethyl (CNET) impurities.

It was found that the object could be achieved with the process for the production of a linear P-linked oligonucleotide bearing an alkyl phosphonate group at the 5′-terminus of the formula I

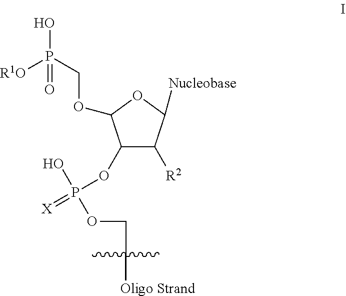

wherein R1 is a C1-4-alkyl group, R2 is fluoro, hydroxyl or C1-4-alkoxy, X is sulfur or oxygen, the term Nucleobase stands for an optionally modified adenine, cytosine, guanine or uracil and the term oligo strand stands for the remaining P-linked oligonucleotide strand, and wherein the oligo strand comprises at least one uracil nucleobase,
which comprises the removal of the cyanoethyl group and one C1-4 alkyl group from the oligonucleotide compound of formula II

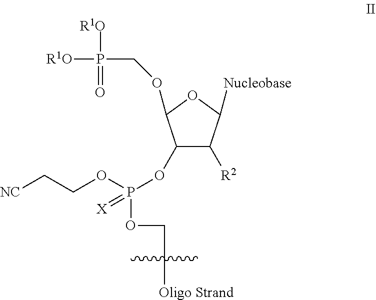

wherein R¹, R², X, the term Nucleobase and the term oligo strand is as above,
with a nucleophilic organic base in the presence of an organic solvent.

The following definitions are set forth to illustrate and define the meaning and scope of the various terms used to describe the invention herein.

The term Nucleobase stands for an optionally modified adenine, cytosine, guanine or uracil. In some embodiments, Nucleobase is uracil.

The term C1-4-alkyl stands for a linear or branched alkyl group of 1 to 4 C-atoms. Representatives are methyl, ethyl, n-propyl, i-propyl, n-butyl, i-butyl or t-butyl. Preferable C1-4-alkyl group for the purpose of the present invention are methyl and ethyl, more preferably methyl.

The term C1-4-alkoxy stands for a linear or branched alkyl group of 1 to 4 C-atoms which is covalently bound to an oxygen atom. Representatives are methoxy, ethoxy, n-propoxy, i-propoxy, n-butoxy, i-butoxy or t-butoxy. Preferable C1-4-alkoxy group for the purpose of the present invention are methoxy and ethoxy, more preferably methoxy.

The term oligonucleotide as used herein is defined as it is generally understood by the skilled person as a molecule comprising two or more covalently linked nucleotides. For use as a therapeutically valuable oligonucleotide, oligonucleotides are typically synthesized as 10 to 40 nucleotides, preferably 10 to 25 nucleotides in length.

The oligonucleotides may consist of optionally modified DNA, RNA or LNA nucleoside monomers or combinations thereof.

The LNA nucleoside monomers are modified nucleosides which comprise a linker group or a bridge between C2′ and C4′ of the ribose sugar ring of a nucleotide. These nucleosides are also termed bridged nucleic acid or bicyclic nucleic acid (BNA) in the literature.

Optionally modified as used herein refers to nucleosides modified as compared to the equivalent DNA, RNA or LNA nucleoside by the introduction of one or more modifications of the sugar moiety or the nucleobase moiety. In a preferred embodiment the modified nucleoside comprises a modified sugar moiety, and may for example comprise one or more 2′ substituted nucleosides and/or one or more LNA nucleosides. The term modified nucleoside may also be used herein interchangeably with the term “nucleoside analogue” or modified “units” or modified “monomers”.

The DNA, RNA or LNA nucleosides are as a rule linked by a phosphodiester (P═O) and/or a phosphorothioate (P═S) internucleoside linkage which covalently couples two nucleosides together.

Accordingly, in some oligonucleotides all internucleoside linkages may consist of a phosphodiester (P═O), in other oligonucleotides all internucleoside linkages may consist of a phosphorothioate (P═S) or in still other oligonucleotides the sequence of internucleoside linkages vary and comprise both phosphodiester (P═O) and phosphorothioate (P═S) internucleoside.

The oligonucleotide constitution may be indicated from 5′-end (left) to 3′-end (right) using a three letter code with each nucleotide being described by three letters where
the first letter defines the sugar moiety (f=2′-fluoro-2′-deoxyribonucleotide, g=2′-modified GalNAc ribonucleotide, m=2′-O-methyl ribonucleotide, p=4′-O-(methylphosphonate-2′-O-methyl ribonucleotide isostere),
the second letter defines the nucleobase (A=adenine, C=cytosine, G=guanine, U=uracil)
the third letter defines the phosphor backbone (o=phosphodiester, s=phosphorothioate)

The term modified nucleobases include but are not limited to nucleobases carrying protecting groups and can be selected from tert·butylphenoxyacetyl, phenoxyacetyl, benzoyl, acetyl, isobutyryl or dimethylformamidino (see Wikipedia, Phosphoramidit-Synthese, https://de.wikipedia.org/wiki/Phosphoramidit-Synthese of Mar. 24, 2016).

The principles of the oligonucleotide synthesis are well known in the art (see e.g. Oligonucleotide synthesis; Wikipedia, the free encyclopedia; https://en.wikipedia.org/wiki/Oligonucleotide synthesis, of Mar. 15, 2016).

Larger scale oligonucleotide synthesis nowadays is carried automatically using computer-controlled synthesizers.

As a rule, oligonucleotide synthesis is a solid-phase synthesis methodology, wherein the oligonucleotide being assembled is covalently bound, via its 3′-terminal hydroxy group, to a solid support material and remains attached to it over the entire course of the chain assembly. Suitable supports are the commercial available macroporous polystyrene supports such as Primer support 5G from Cytiva and NittoPhase® HL support from Kinovate, or controlled pore glass supports like the nucleobase pre-loaded CPG support supplied by LGC.

As outlined above the oligonucleotide synthesis in principle is a stepwise addition of nucleoside residues to the 5′-terminus of the growing chain until the desired sequence is assembled as outlined above.

The backbone deprotection can be performed in accordance with the process of the present invention as outlined below. The subsequent cleavage from the resin can be performed with concentrated aqueous ammonia.

In a preferred embodiment the present invention comprises the process for the production of a linear P-linked oligonucleotide bearing an alkyl phosphonate group at the 5′-terminus of the formula Ia

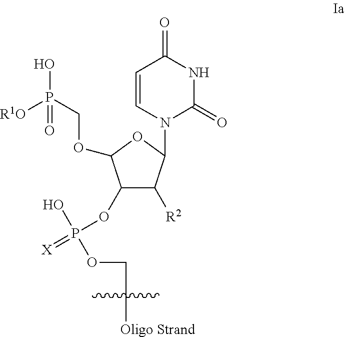

wherein R1 is a C1-4-alkyl group, R2 is fluoro, hydroxyl or C1-4-alkoxy, X is sulfur or oxygen and oligo strand stands for the remaining P-linked oligonucleotide strand.

The starting compound of the process of the present invention, the oligonucleotide compound of formula II, can be prepared in accordance with the disclosure of the PCT International Publication WO 2018/045317.

In a preferred embodiment the oligonucleotide compound of formula II has the formula IIa

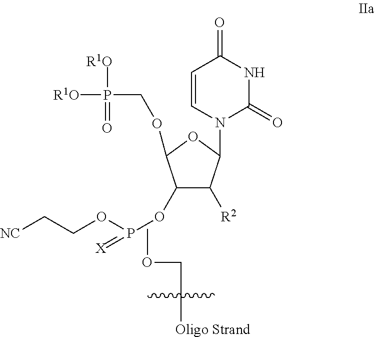

As outlined above R1 is a C1-4-alkyl group, R2 is fluoro, hydroxyl or C1-4-alkoxy. In a preferred embodiment R1 is methyl and R2 is fluoro, hydroxyl or methoxy. In a more preferred embodiment R1 is methyl and R2 is fluoro or methoxy.

The nucleophilic organic base typically is an organic amine which can be characterized by its nucleophilicity in accordance with the Mayr nucleophilicity scale: J. Phys. Org. Chem. 2008, 21, 584-595) and its pKa in accordance with Wikipedia, Acid dissociation constant, https://en.wikipedia.org/wiki/Acid_dissociation_constant of Oct. 24, 2021).

Usually the nucleophilic organic base has nucleophilicity of higher than 15, preferably between 15 and 30, more preferably between 16 and 25.

The pKa value of suitable nucleophilic organic bases base (protonated organic base) is less than 10.5, preferably between 6 and 10.5, more preferably between 8 and 10.5.

Preferred nucleophilic organic bases are tertiary amines which can be selected from morpholine, N,N-dimethylethylamine, N-methyl-pyrrolidine or from (1,4-diazabicyclo[2.2.2]octane.

Preferred nucleophilic organic base is (1,4-diazabicyclo[2.2.2]octane) (DABCO).

The process requires the presence of an organic solvent, which can be selected from acetonitrile, pyridine and toluene or from mixtures thereof. Preferred organic solvent is acetonitrile.

The concentration of the nucleophilic organic base in the organic solvent is as a rule selected in the range of 5% (w) and 100% (w), preferably 10% (w) to 50% (w), more preferably 15% (w) to 25% (w).

The amount of the nucleophilic organic base is typically applied in a range of 1.5 CV to 30.0 CV.

The solution of the nucleophilic organic base in the organic solvent is usually delivered in a time range of 10 min to 6 hours, preferably 20 min to 4 h.

A flow rate in the range of 0.1 CV/min to 2.0 CV/min, preferably 0.1 CV/min to 0.5 CV/min was found to be practicable.

After complete delivery, the solution of the nucleophilic organic base in the organic solvent can be recycled over the synthesis column for a time between 60 min and 4 hours, preferably 90 min to 120 min. This is a further preferred embodiment as it allows, if required, to prolong the reaction time without the need to add fresh organic base.

As a typical example, 3.75 CV of the nucleophilic organic base in acetonitrile is charged to the column over 30 min followed by recycling over the column for 90 min at 0.125 CV/min.

As mentioned above the object of the present intention was suppressing or at least reducing unwanted formation of alkyl transfer impurities and of cyanoethyl (CNET) impurities because the alkyl group (R1) has a tendency to alkylate uracil nucleobases in proximity to the alkyl phosphonate nucleotide, which results in so called alkyl transfer impurities as illustrated in the oligo structure of formula III below with a methylated uracil nucleobase.

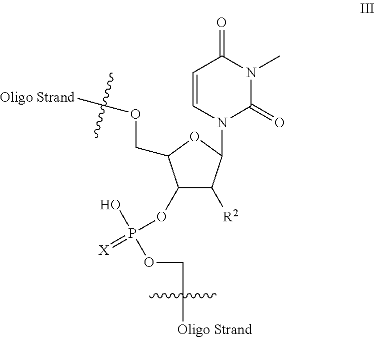

These impurities cannot be significantly depleted in the subsequent downstream processing steps.

As the alkyl transfer impurities or preferably the methyl transfer impurities may affect uracil nucleobases of the oligo strand in the oligonucleotide compound of formula II or IIa and in the resulting linear P-linked oligonucleotide of the formula I or Ia, the oligonucleotide in some embodiments contains at least one uracil nucleobase.

Since the tendency to alkylate the uracil nucleobase and that the alkyl transfer impurities increase the closer the uracil nucleobases are located on the oligo strand to the 5′ terminus, the term proximity means that the uracil nucleobases are typically within the 2nd to the 20th position, preferably within the 2nd to the 10th position, more preferably within the 2nd and the 6th position of the oligo strand, counted from the 5′ terminus.

The process of the present invention, as a further embodiment, therefore comprises performing the process under conditions that the level of alkyl transfer impurities, expressed as “sum of N+alkyl impurities”, in the linear P-linked oligonucleotide of formula I is below 4.0%, below 3.0%, below 2.0%, below 1.0%, or most preferred below 0.5%. The % values are “% area” determined from the % area of the UV peaks, which is corrected by MS intensity of the N+alkyl impurity group.

In a preferred embodiment the N+alkyl impurities are N+methyl impurities.

The process of the present invention as a further embodiment also comprises performing the process under conditions that the level of cyanoethyl (CNET) impurities, expressed as “sum of CNET impurities”, in the linear P-linked oligonucleotide of formula I, is below 2.0%, below 1.0% or below 0.5%. The % values are “% area” determined from the % area of the UV peaks, which is corrected by MS intensity of the CNET impurity group.

These values can be achieved and measured at the crude oligonucleotide stage, i.e. for the oligonucleotide obtained after cleavage and deprotection and before any downstream processing like purification or ultrafiltration is applied.

By way of illustration the oligonucleotide can be selected from:

(SEQ ID NO: 2)
pUs.fUs.fAs.mUo.fUo.mGo.fUo.fGo.mAo.fGo.mGo.
fAo.mUo.fUo.mUo.fUo.mUo.mGo.fUo.mCs.mGs.mG

wherein the oligonucleotide constitution is indicated from 5′-end (left) to 3′-end (right) using a three-letter code with each nucleotide being described by three letters where
the first letter defines the sugar moiety (f=2′-fluoro-2′-deoxyribonucleotide, g=2′-modified GalNAc ribonucleotide, m=2′-O-methyl ribonucleotide, p=methyl-phosphonate-2′-O-methyl ribonucleotide isostere),
the second letter defines the nucleobase (A=adenine, C=cytosine, G=guanine, U=uracil)
the third letter defines the phosphor backbone (o=phosphodiester, s=phosphorothioate)

The compounds disclosed herein have the following nucleobase sequences

SEQ ID No. 1:
UUAUUGUGAGGAUUUUUGUCGG

### EXAMPLES

### Abbreviations

Ac₂O=acetic acid anhydride
ETT=5-ethylthiotetrazol
Bz=benzoyl
CNET=cyanoethyl
DABCO=1,4-diazabicyclo[2.2.2]octane
DCA=dichloroacetic acid
DEA=diethylamine
DNA=2′-deoxyribonucleotide
DMEA=N′N dimethylethylamine
DMT=4,4′-dimethoxytrityl
CV=column volume
MeCN=acetonitrile
NA=not applicable
NMI=N-methyl imidazole
NMP=N-methyl-pyrrolidine
PhMe=Toluene
TBA=tert-butylamine
PhMe=Toluene

### Example 1

### Synthesis of

(SEQ ID NO: 3)
pUs.fUs.fAs.mUo.fUo.mGo.fUo.fGo.mAo.fGo.mGo.
fAo.mUo.fUo.mUo.fUo.mUo.mGo.fUo.mCs.mGs.mG

Wherein the oligonucleotide constitution is indicated from 5′-end (left) to 3′-end (right) using a three letter code with each nucleotide being described by three letters where
the first letter defines the sugar moiety (f=2′-fluoro-2′-deoxyribonucleotide, g=2′-modified GalNAc ribonucleotide, m=2′-O-methyl ribonucleotide, p=4′-O-(methylphosphonate-2′-O-methyl ribonucleotide isostere),
the second letter defines the nucleobase (A=adenine, C=cytosine, G=guanine, U=uracil)
the third letter defines the phosphor backbone (o=phosphodiester, s=phosphorothioate)

The title compound was produced by standard phosphoramidite chemistry on solid phase at a scale of 2.62 mmol using an AKTA Oligopilot 100 and preloaded polystyrene solid support (NittoPhase HL preloaded 358).

The following phosphoramidites have been used in each cycle:

| Cycle | P-amidite |
|---|---|
| 1 | mG(iBu) |
| 2 | mC(Ac) |
| 3 | fU |
| 4 | mG(iBu) |
| 5 | mU |
| 6 | fU |
| 7 | mU |
| 8 | fU |
| 9 | mU |
| 10 | fA(Bz) |
| 11 | mG(iBu) |
| 12 | fG(iBu) |
| 13 | mA(Bz) |
| 14 | fG(iBu) |
| 15 | fU |
| 16 | mG(iBu) |
| 17 | fU |
| 18 | mU |
| 19 | fA(Bz) |
| 20 | fU |
| 21 | pU |

In general 2.0 equiv of the phosphoramidites were employed. All reagents were used as received from commercially available sources and reagent solutions at the appropriate concentration were prepared (see details below). Cleavage and deprotection was achieved using ammonium hydroxide to give the crude oligonucleotide.

### Standard Reagent Solutions

| | |
|---|---|
| Deblock | 3 vol % DCA in toluene |
| Phosphoramidites | 0.2 M in acetonitrile for mA(Bz), |
| | mC(Ac), mG(iBu), fA(Bz), |
| | fC(Ac), fG(iBu), fU, pU |
| | 0.2 M in acetonitrile/toluene |
| | (85:15, v/v) for mU |
| Activator | 0.6 M ETT in acetonitrile |
| Thiolation | 0.2 M Xanthanhydride in pyridine |
| Cap A | NMI/2,6-lutidine/acetonitrile: 20/30/50 |
| Cap B | 20 vol % acetic anhydride in acetonitrile |
| Backbone deprotection | See experiments |
| Cleavage and | 28-32% aqueous ammonium |
| Deprotection | hydroxide at 35° C. |

The crude solution from the cleavage & deprotection step was concentrated in vacuo to remove excess ammonia. The concentrated solution was lyophilized to provide the crude oligonucleotide as a solid. The pale-yellow solid was sampled and submitted to LC-UV-MS analysis. Impurities were grouped according to their assigned structure. The sum of all N+Methyl impurities and the sum of all CNET impurities were used for the analysis of the process parameters.

### Example 2

### Backbone Deprotection Examples

The organic amines used have the pKa and nucleophilicity values listed in the table below. The nucleophilicity values can be found in Mayr's Database of Reactivity Parameters (https://www.cup.lmu.de/oc/mayr/reaktionsdatenbank/fe/showclass/40) and the pKa values in “Correlation of the Base Strengths of Amines, J. Am. Chem. Soc. 1957, 79, 20, 5441-5444». The pKa of DABCO is referenced in the “Basicity of 1,s-bis(dimethylamino)-naphthalene and 1,4-diazabicyclo[2.2.2]octane in water and dimethylsulfoxide, Can. J. Chem. 1987, 65, 996.

| Amine | pKa | Nucleophilicity |
|---|---|---|
| DABCO | 8.82 | 18.80 |
| DEA | 10.98 | 15.10 |
| DMEA | 9.99 | N/A |
| Morpholine | 8.36 | 15.65 |
| NMP | 10.46 | 20.59 |
| TBA | 10.45 | 12.35 |
| TEA | 10.65 | 17.10 |

Backbone deprotection was performed using various solutions of organic amines in toluene, pyridine or acetonitrile as outlined in the table below.

| | | | | Sum of | Sum of | |
|---|---|---|---|---|---|---|
| | Nucleophilic | | Concentration | N + methyl | CNET | |
| | organic base | | Nob in solvent | impurities | impurities | Yield |
| Example | (Nob) | Solvent | [weight l %] | [area %] | [area %] | [%] |
| 2a) | TBA | MeCN | 18.1 | 5.6 | 0.5 | 72 |
| comparison | | | | | | |
| 2b) | TBA | MeCN | 18.1 | 8.3 | 0.9 | 70 |
| comparison | | | | | | |
| 2c) | DEA | MeCN | 18.4 | 5.2 | 0.3 | 71 |
| comparison | | | | | | |
| 2d) | TEA | MeCN | 18.8 | 4.2 | 0.2 | 72 |
| comparison | | | | | | |
| 2e) | Morpholine | MeCN | 24.1 | 1.7 | 0.1 | 71 |
| 2f) | Morpholine | MeCN | 45.8 | 1.6 | 0.1 | 54 |
| 2g) | Morpholine | — | 100 | 0.9 | 0.1 | 59 |
| 2h) | Morpholine | Pyridine | 20.2 | 2.5 | 0.1 | 67 |
| 2i) | Morpholine | Toluene | 22.3 | 2.3 | 0.2 | 70 |
| 2j) | DABCO | MeCN | 20 | 0.3 | 0.1 | 70 |
| 2k) | DABCO | MeCN | 20 | 0.3 | 0.1 | 70 |
| 2l) | DMEA | MeCN | 17.7 | 2.2 | 0.1 | 72 |
| 2m) | NMP | MeCN | 20.7 | 1.7 | 0.3 | 70 |

The process parameters are listed the separate table below.

| | Nucleophilic | | Amount of | | | |
|---|---|---|---|---|---|---|
| | organic base | | Nob in solvent | Delivery | Recycling | Yield |
| Example | (Nob) | Solvent | [CV] | time [min] | time [min] | [%] |
| 2a) | TBA | MeCN | 5.00 | 45 | — | 72 |
| comparison | | | | | | |
| 2b) | TBA | MeCN | 26.67 | 240 | — | 70 |
| comparison | | | | | | |
| 2c) | DEA | MeCN | 26.67 | 240 | — | 71 |
| comparison | | | | | | |
| 2d) | TEA | MeCN | 26.67 | 240 | — | 72 |
| comparison | | | | | | |
| 2e) | Morpholine | MeCN | 26.67 | 240 | — | 71 |
| 2f) | Morpholine | MeCN | 26.67 | 240 | — | 54 |
| 2g) | Morpholine | — | 5.00 | 45 | 195 | 59 |
| 2h) | Morpholine | Pyridine | 26.67 | 240 | — | 67 |
| 2i) | Morpholine | Toluene | 26.67 | 240 | — | 70 |
| 2j) | DABCO | MeCN | 26.67 | 240 | — | 70 |
| 2k) | DABCO | MeCN | 3.75 | 30 | 90 | 70 |
| 2l) | DMEA | MeCN | 26.67 | 240 | — | 72 |
| 2m) | NMP | MeCN | 26.67 | 240 | — | 70 |

Examples 2e, 2j, 2k, 2l and 2m are regarded as preferred. Example 2k is most preferred.

1. Process for the production of a linear P-linked oligonucleotide bearing an alkyl phosphonate group at the 5′-terminus of the formula 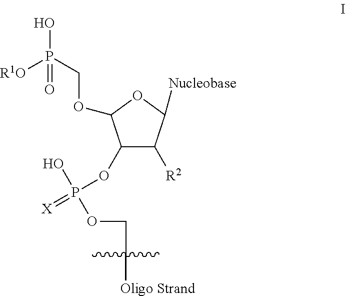
wherein R¹ is a C₁₋₄-alkyl group, R² is fluoro, hydroxyl or C₁₋₄-alkoxy, X is sulfur or oxygen, the term Nucleobase stands for an optionally modified adenine, cytosine, guanine or uracil and the term oligo strand stands for the remaining P-linked oligo nucleotide strand, and wherein the oligo strand comprises at least one uracil nucleobase, comprising the removal of the cyanoethyl group and one C₁₋₄ alkyl group from the oligonucleotide compound of formula II 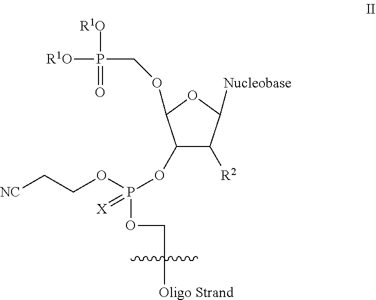
wherein R¹, R², X, the term Nucleobase and the term oligo strand is as above, with a nucleophilic organic base in the presence of an organic solvent.

2. Process of claim 1, wherein R¹ is methyl.

3. Process of claim 1, wherein R² is fluoro or methoxy.

4. Process of claim 1, wherein the Nucleobase is adenine, cytosine, guanine, or uracil.

5. Process of claim 1, wherein the nucleophilic organic base has a nucleophilicity of higher than 15.

6. Process of claim 1, wherein the nucleophilic organic base has a pKa of the protonated organic base of less than 10.5.

7. Process of claim 1, wherein the nucleophilic organic base is a tertiary amine selected from morpholine, N,N-dimethylethylamine, N-methyl-pyrrolidine or 1,4-diazabicyclo[2.2.2]octane.

8. Process of claim 1, wherein the nucleophilic organic base is (1,4-diazabicyclo[2.2.2]octane) (DABCO).

9. Process of claim 1, wherein the organic solvent is selected from acetonitrile, pyridine and toluene or from mixtures thereof.

10. Process of claim 1, wherein the organic solvent is acetonitrile.

11. Process of claim 1, wherein the concentration of the nucleophilic organic base in the organic solvent is selected in the range of 5% (w) and 100% (w).

12. Process of claim 1, wherein the amount of the nucleophilic organic base is applied in a range of 1.5 CV to 30.0 CV and the flow rate is selected in the range of 0.1 CV/min to 2.0 CV/min.

13. Process of claim 1, wherein the process is performed under conditions that the level of alkyl transfer impurities, expressed as “sum of N+alkyl impurities”, in the linear P-linked oligonucleotide of formula I, is below 4.0%.

14. Process of claim 1, wherein the process is performed under conditions that the level cyanoethyl (CNET) impurities, expressed as “sum of CNET impurities”, in the linear P-linked oligonucleotide of formula I, is below 2.0%.

15. Process of claim 5, wherein the nucleophilic organic base as a nucleophilicity of between 16 and 25.

16. Process of claim 6, wherein the nucleophilic organic base has a pKa of the protonated organic base of between 8 and 10.5.

17. Process of claim 11, wherein the concentration of the nucleophilic organic base in the organic solvent is selected in the range of 15% (w) to 25% (w).

18. Process of claim 12, wherein the amount of the nucleophilic organic base is applied in a range of 1.5 CV to 30.0 CV and the flow rate is selected in the range of 0.1 CV/min to 0.5 CV/min.

19. Process of claim 13, wherein the process is performed under conditions that the level of alkyl transfer impurities, expressed as “sum of N+alkyl impurities”, in the linear P-linked oligonucleotide of formula I, is below 0.5%.

20. Process of claim 14, wherein the process is performed under conditions that the level cyanoethyl (CNET) impurities, expressed as “sum of CNET impurities”, in the linear P-linked oligonucleotide of formula I, is below 0.5%.


SEQUENCE LISTING (3 sequences)

SEQ ID NO: 1 - RNA, 22 nt, synthetic construct
  source 1..22: mol_type = other RNA
ttattgtgaggatttttgtcgg

SEQ ID NO: 2 - RNA, 22 nt, synthetic construct
  source 1..22: mol_type = other RNA
  modified_base 1: mod_base = OTHER; note = methyl-phosphonate-2'-O-methyl ribonucleotide isostere with phosphorothioate linkage
  modified_base 2: mod_base = OTHER; note = 2'-fluoro-2'-deoxyribonucleotide with phosphorothioate linkage
  modified_base 3: mod_base = OTHER; note = 2'-fluoro-2'-deoxyribonucleotide with phosphorothioate linkage
  modified_base 4: mod_base = OTHER; note = 2'-O-methyl ribonucleotide
  modified_base 5: mod_base = OTHER; note = 2'-fluoro-2'-deoxyribonucleotide
  modified_base 6: mod_base = OTHER; note = 2'-O-methyl ribonucleotide
  modified_base 7: mod_base = OTHER; note = 2'-fluoro-2'-deoxyribonucleotide
  modified_base 8: mod_base = OTHER; note = 2'-fluoro-2'-deoxyribonucleotide
  modified_base 9: mod_base = OTHER; note = 2'-O-methyl ribonucleotide
  modified_base 10: mod_base = OTHER; note = 2'-fluoro-2'-deoxyribonucleotide
  modified_base 11: mod_base = OTHER; note = 2'-O-methyl ribonucleotide
  modified_base 12: mod_base = OTHER; note = 2'-fluoro-2'-deoxyribonucleotide
  modified_base 13: mod_base = OTHER; note = 2'-O-methyl ribonucleotide
  modified_base 14: mod_base = OTHER; note = 2'-fluoro-2'-deoxyribonucleotide
  modified_base 15: mod_base = OTHER; note = 2'-O-methyl ribonucleotide
  modified_base 16: mod_base = OTHER; note = 2'-fluoro-2'-deoxyribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-fluoro-2'-deoxyribonucleotide
  modified_base 20: mod_base = OTHER; note = 2'-O-methyl ribonucleotide with phosphorothioate linkage
  modified_base 21: mod_base = OTHER; note = 2'-O-methyl ribonucleotide with phosphorothioate linkage
  modified_base 22: mod_base = OTHER; note = 2'-O-methyl ribonucleotide
ttattgtgaggatttttgtcgg

SEQ ID NO: 3 - RNA, 22 nt, synthetic construct
  source 1..22: mol_type = other RNA
  modified_base 1: mod_base = OTHER; note = 4'-O-(methylphosphonate-2'-O-methyl ribonucleotide isostere) with phosphorothioate linkage
  modified_base 2: mod_base = OTHER; note = 2'-fluoro-2'-deoxyribonucleotide with phosphorothioate linkage
  modified_base 3: mod_base = OTHER; note = 2'-fluoro-2'-deoxyribonucleotide with phosphorothioate linkage
  modified_base 4: mod_base = OTHER; note = 2'-O-methyl ribonucleotide
  modified_base 5: mod_base = OTHER; note = 2'-fluoro-2'-deoxyribonucleotide
  modified_base 6: mod_base = OTHER; note = 2'-O-methyl ribonucleotide
  modified_base 7: mod_base = OTHER; note = 2'-fluoro-2'-deoxyribonucleotide
  modified_base 8: mod_base = OTHER; note = 2'-fluoro-2'-deoxyribonucleotide
  modified_base 9: mod_base = OTHER; note = 2'-O-methyl ribonucleotide
  modified_base 10: mod_base = OTHER; note = 2'-fluoro-2'-deoxyribonucleotide
  modified_base 11: mod_base = OTHER; note = 2'-O-methyl ribonucleotide
  modified_base 12: mod_base = OTHER; note = 2'-fluoro-2'-deoxyribonucleotide
  modified_base 13: mod_base = OTHER; note = 2'-O-methyl ribonucleotide
  modified_base 14: mod_base = OTHER; note = 2'-fluoro-2'-deoxyribonucleotide
  modified_base 15: mod_base = OTHER; note = 2'-O-methyl ribonucleotide
  modified_base 16: mod_base = OTHER; note = 2'-fluoro-2'-deoxyribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-fluoro-2'-deoxyribonucleotide
  modified_base 20: mod_base = OTHER; note = 2'-O-methyl ribonucleotide with phosphorothioate linkage
  modified_base 21: mod_base = OTHER; note = 2'-O-methyl ribonucleotide with phosphorothioate linkage
  modified_base 22: mod_base = OTHER; note = 2'-O-methyl ribonucleotide
ttattgtgaggatttttgtcgg